SEQUENCE LISTING (5 sequences)

SEQ ID NO: 1 - protein, 20 aa, synthetic construct
MEINEKLLRQIIEDVLRDMK

SEQ ID NO: 2 - protein, 18 aa, synthetic construct
MNTSELETLIRTILSEQL

SEQ ID NO: 3 - protein, 20 aa, synthetic construct
MDKELLQSTVRKVLDEMRQR

SEQ ID NO: 4 - protein, 23 aa, synthetic construct
MNGETLQRIVEEIVSRLHRRAQS

SEQ ID NO: 5 - protein, 22 aa, synthetic construct
MSSNELVEQIMAQVIARVATPE

(19) United States
(12) Patent Application Publication
(10) Pub. No.: US 2026/0132409 A1
(43) Pub. Date: May 14, 2026

(54) SIGNAL SEQUENCES FOR DELIVERING CARGO TO BACTERIAL MICROCOMPARTMENTS

(71) Applicant: Northwestern University, Evanston, IL (US)

(72) Inventors: Danielle Tullman-Ercek, Evanston, IL (US); Nolan Warren Kennedy, Evanston, IL (US); Elizabeth Ruth Johnson, Evanston, IL (US); Carolyn Elaine Mills, Evanston, IL (US)

(21) Appl. No.: 19/118,278

(22) PCT Filed: Oct. 5, 2023
(86) PCT No.: PCT/US2023/076100

§ 371 Date: Apr. 3, 2025

Related U.S. Application Data
(60) Provisional application No. 63/378,508, filed on Oct. 5, 2022.

(51) Int. Cl. C12N 15/74; C07K 14/255; C12N 1/20; C12R 1/42

(52) U.S. Cl. CPC C12N 15/74; C07K 14/255; C12N 1/20; C07K 2319/02; C12R 2001/42

(57) ABSTRACT

The present disclosure provides fusion proteins comprises fusion proteins comprising the signal sequence of PduB or PduM and a heterologous protein, as well as constructs for expressing the fusion proteins, and methods of their use. The fusion proteins are designed to deliver the heterologous proteins to bacterial microcompartments and modify the 1,2-propanediol metabolic pathway.

### CROSS-REFERENCE TO RELATED APPLICATIONS

This application claims priority to U.S. Provisional Application No. 63/378,508 filed on Oct. 5, 2022, the content of which is incorporated by reference in its entirety.

### STATEMENT REGARDING FEDERALLY SPONSORED RESEARCH

This invention was made with government support under DE-SC0022180 awarded by the Department of Energy and W911NF-19-1-0298 awarded by the Department of Defense. The government has certain rights in the invention.

### REFERENCE TO A SEQUENCE LISTING

The contents of the electronic sequence listing (702581.02417.xml; Size: 5,319 bytes; and Date of Creation: Oct. 5, 2023) is herein incorporated by reference in its entirety.

### BACKGROUND OF THE INVENTION

Bacterial microcompartments (MCPs) are proteinaceous organelles present in over half of bacterial phyla which encapsulate enzymatic pathways in a semipermeable protein shell. MCPs could benefit these pathways by sequestering toxic intermediates, internally recycling cofactors, colocalizing enzymes, or isolating intermediates from competing pathways. There is a need to apply these organelles to the encapsulation of engineered enzymatic pathways to enhance pathway flux.

### SUMMARY OF THE INVENTION

In an aspect, provided herein is a fusion protein comprising an N-terminal signal sequence of a Pdu protein selected from PduM and PduB; and a heterologous cargo protein. In embodiments, the signal sequence of PduM is SEQ ID NO: 4; and the signal sequence of PduB is SEQ ID NO: 5. In embodiments, the fusion protein comprises the signal sequence of PduB. In embodiments, the fusion protein comprises the signal sequence of PduM. In embodiments, the heterologous cargo protein comprises an enzyme. In embodiments, the enzyme is an enzyme of the 1,2-propanediol degradation pathway. In embodiments, the heterologous cargo protein comprises a detectable protein.

In another aspect, provided herein is a composition comprising two or more fusion proteins disclosed herein; wherein each fusion protein comprises a different heterologous cargo protein. In embodiments, the fusion protein comprises the same signal sequence. In embodiments, each fusion protein comprises a different signal sequence.

In another aspect, provided herein is a construct configured to express any of the fusion proteins disclosed herein.

In another aspect, provided herein is a bacterial microcompartment comprising at least one of the fusion proteins disclosed herein. In embodiments, the microcompartment further comprises endogenous PduM and endogenous PduB. In embodiments, the microcompartment further comprises at least one of endogenous PduD, PduP, and PduL. In embodiments, the microcompartment comprises at least one of the signal sequences of PduD, PduP, and PduL. In embodiments, the microcompartment is a 1,2-propanediol microcompartment.

In another aspect, provided herein is a host cell comprising at least one of the fusion proteins disclosed herein, the composition disclosed herein, the construct disclosed herein, or the bacterial microcompartment disclosed herein.

In another aspect, provided herein is a kit comprising at least two of the fusion proteins disclosed herein, wherein each fusion protein comprises a different heterologous cargo protein, and wherein each fusion protein is separate from the others. In embodiments, each fusion protein comprises the same signal sequence. In embodiments, each fusion protein comprises a different signal sequence.

In another aspect, provided herein is a method of targeting a microcompartment in a cell, the method comprising expressing at least one of the fusion proteins disclosed herein in the cell. In embodiments, the microcompartment is a 1,2-propanediol microcompartment. In embodiments, the fusion protein modifies 1,2-propanediol degradation, and wherein the fusion protein comprises any one of the fusion proteins disclosed herein. In embodiments, the microcompartment is an ethanolamine utilization (Eut) microcompartment. In embodiments, the cell is a Salmonella enterica cell.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

This patent or application file contains at least one drawing executed in color. Copies of this patent or patent application publication with color drawing(s) will be provided by the Office upon request and payment of the necessary fee.

FIGS. 1A-1C illustrate the pdu operon and MCP structure. (A) Native pdu pathway for 1,2-propanediol degradation, including main pathway and cofactor recycling enzymes. (B) Transmission electron microscopy (TEM) of purified Pdu MCPs. (C) pdu operon and adjacent genes.

FIG. 2 illustrates signal sequence encapsulation efficiency. ssPduD has the highest encapsulation efficiency, followed by ssPduP, then ssPduL.

FIG. 3 illustrates an alignment of Pdu signal sequences (ssPduD (SEQ ID NO: 1), ssPduP (SEQ ID NO: 2), ssPduL (SEQ ID NO: 3), ssPduM (SEQ ID NO: 4), ssPduB (SEQ ID NO: 5)). Signal sequences share a common motif of alternating hydrophobic (red) and hydrophilic (blue) residues.

FIG. 4 illustrates structures of Pdu signal sequences. The signal sequences share a common structure. Hydrophobicity surfaces of the Pdu signal sequences. Structures were generated by AlphaFold and visualized by UCSF Chimera. Hydrophobic areas are shown in red and hydrophilic areas are shown in blue.

FIG. 5 shows average puncta per cell in signal sequence knockout strains as proportions of wild-type puncta count for all encapsulation reporters. Average puncta count decreases in signal sequence knockout strains.

FIG. 6 shows TEM images of purified MCPs from enzymatic signal sequence knockout strains. Enzymatic signal sequence knockout strains form MCP shells but show slight disruptions in morphology.

FIG. 7 shows fluorescence microscopy of PduM¹⁻²²-GFP, PduB¹⁻³⁷-GFP (signal sequence+linker), and PduB¹⁻²²-GFP in WT, ΔPduB, and ΔpocR S. enterica. PduM¹⁻²² and PduB¹⁻²² act as signal sequences to target GFP to the MCP core.

FIG. 8 shows fluorescence microscopy of all encapsulation reporters in all enzymatic signal sequence knockout strains. Scale bars are 1 μm.

FIG. 9 shows fluorescence microscopy of GFP constructs in PduM, PduB, and structural signal sequence knockout strains. Scale bars are 1 μm.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

Bacterial microcompartments (MCPs) are organelle-like structures having a protein shell that encloses a core comprising enzymes and other proteins. The shell functions like a membrane that is selectively permeable. MCP-encapsulated metabolosomes have been found in 45 of 83 bacterial phyla and metabolize a variety of substrates. Despite the diversity in substrates, encapsulated pathways share many similar characteristics. The first encapsulated step is typically catalyzed by a signature enzyme that converts the substrate to a toxic, volatile aldehyde intermediate. This is followed by an aldehyde dehydrogenase (AldDH) that detoxifies the aldehyde to form an acyl-CoA product, then several other steps that convert the acyl-CoA to a compound that can be used by central metabolism. As a result, most MCPs encapsulate similar sets of enzymes. In addition to a signature enzyme, 94% of MCP loci contain an AldDH gene, 76% have an alcohol dehydrogenase that regenerates NAD⁺ for the AldDH, and 66% contain a phosphotransacylase. The most common and best characterized MCP systems are the 1,2-propanediol utilization (Pdu) and ethanolamine utilization (Eut) MCPs, which are commonly found in enteric pathogens. Both Pdu and Eut MCPs have B₁₂-dependent signature enzymes and consume niche carbon sources found in the gut. Other MCP systems have been identified that are predicted to metabolize ethanol, rhamnose, fucose, sugar phosphates from DNA degradation, and aromatic compounds.

The propanediol utilizing (PDU) MCP breaks down 1,2-propanediol to propanol and propionyl-phosphate, which is then dephosphorylated to propionate, generating ATP. The PDU MCP is typically encoded by a 21 gene locus, including genes that encode structural proteins of the shell and genes that encode proteins of the enzymatic core. Some intermediates in the 1,2-propanediol metabolic pathway are toxic and must be sequestered to prevent cell damage.

The N-terminal ends of many MCP proteins include encapsulation peptides, or signal sequences, which play a role in targeting them to the core. Numerous signal sequences have been identified and characterized in the model PDU microcompartment from Salmonella enterica serovar Typhimurium LT2. The inventors examined previously identified signal sequences and found that they are important for targeting their associated enzymes, as well as other enzymes that do not have signal sequences to the MCP core. The inventors also identified previously uncharacterized signal sequences on structural proteins PduM and PduB, which are important for coordinating assembly of the MCP core. These signal sequences may be useful in introducing heterologous proteins to MCPs to modify enzymatic pathways and enhance production of biochemicals.

In a first aspect, the present disclosure provides a fusion protein comprising an N-terminal signal sequence of a Pdu protein selected from PduM and PduB; and a heterologous cargo protein.

The term “fusion protein” refers to a protein or polypeptide formed from the combination of two different proteins or protein fragments. The terms “peptide,” “polypeptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The terms polypeptide, peptide, and protein are also inclusive of modifications including, but not limited to, glycosylation, lipid attachment, sulfation, carboxylation, hydroxylation, ADP-ribosylation, and addition of other complex polysaccharides. The terms “residue” or “amino acid residue” or “amino acid” are used interchangeably to refer to an amino acid that is incorporated into a peptide, protein, or polypeptide. The amino acid may be a naturally occurring amino acid and, unless otherwise limited, may encompass non-natural analogues of natural amino acids that can function in a similar manner as naturally occurring amino acids.

The N-terminal signal sequence of PduM may comprise or consist of SEQ ID NO: 4, or a sequence having at least 80%, 81%, 82%, 83%, 84%, 85%, 86%, 87%, 88%, 89%, 90%, 91%, 92%, 93%, 94%, 95%, 96%, 97%, 98% or 99% identity thereto. The N-terminal signal sequence of PduB may comprise or consist of SEQ ID NO: 5 or a sequence having at least 80%, 81%, 82%, 83%, 84%, 85%, 86%, 87%, 88%, 89%, 90%, 91%, 92%, 93%, 94%, 95%, 96%, 97%, 98% or 99% identity thereto.

As used herein the term “heterologous cargo protein” refers to a protein or polypeptide not endogenous or native to Salmonella enterica serovar Typhimurium LT2. Conversely the terms “endogenous” and “native” are used herein to refer to a protein or polypeptide that naturally occurs in the bacterial cell or strain that it is being introduced or expressed in. The heterologous cargo protein may be an enzyme, e.g. a modified Pdu enzyme or other enzyme that modifies the 1,2-propanediol metabolic pathway. Enzymes that modify the 1,2-propanediol metabolic pathway include, but are not limited to, PduC, PduD, PduE, PduP, PduL, PduG, PduH, PduS, PduO, PduQ, and PduW. The heterologous cargo protein may be a detectable protein, e.g. GFP, etc.

The heterologous cargo protein may be an enzyme that modifies another microcompartment pathway, such as an enzyme that modifies ethanolamine utilization, e.g. ethanolamine-ammonia lyase, aldehyde dehydrogenase (EutE), or alcohol dehydrogenase (EutG), or an enzyme that modifies aldehyde oxidation, e.g. aldehyde dehydrogenase, alcohol dehydrogenase, or phosphotransacylase.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

Two or more fusion proteins described herein may be prepared in a composition for delivery to a cell. Each fusion protein may comprise the same signal sequence or a different signal sequence.

In a second aspect, provided herein is a construct configured to express any of the fusion proteins provided herein.

As used herein, the term “construct” refers to a recombinant polynucleotide, i.e., a polynucleotide that was formed artificially by combining at least two polynucleotide components from different sources (natural or synthetic). For example, the constructs described herein comprise a polynucleotide encoding the fusion protein disclosed herein, operably linked to a promoter that (1) is associated with another gene found within the same genome, (2) from the genome of a different species, or (3) is synthetic. Constructs can be generated using conventional recombinant DNA methods.

The terms “nucleic acid,” “nucleic acid sequence,” “polynucleotide,” and “polynucleotide sequence,” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell. The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

Also provided herein are vectors for expressing the fusion protein in bacteria cells. The vector may be a recombinant vector (e.g., a recombinant expression vector) comprising the nucleic acid sequence or construct encoding the fusion protein described herein. The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” or “recombinant expression vectors.”

One type of vector is a “plasmid”, which refers to a circular double stranded DNA loop into which additional DNA segments may be ligated, specifically exogenous DNA segments encoding the targeted protein. Another type of vector is a viral vector, wherein additional DNA segments may be ligated into the viral genome. Certain vectors are capable of autonomous replication in a host cell into which they are introduced. Moreover, certain vectors are capable of directing the expression of exogenous genes to which they are operatively linked. In general, vectors of utility in recombinant DNA techniques are often in the form of plasmids.

The vectors are heterogeneous exogenous constructs containing sequences from two or more different sources. Suitable vectors include, but are not limited to, plasmids, expression vectors, among others and includes constructs that are able to express the protein of interest in bacterial cells, preferably Salmonella cells. A vector can preferably transduce, transform or infect a cell, thereby causing the cell to express the nucleic acids and/or proteins encoded by the vector.

In a third aspect, provided herein is a bacterial microcompartment comprising at least one of the fusion proteins described herein. The bacterial compartment may be a 1,2-propanediol microcompartment. The bacterial microcompartment may also comprise endogenous PduM and/or PduB. The bacterial microcompartment may also comprise at least one of endogenous PduD, PduP, and PduL. The bacterial microcompartment may comprise all of endogenous PduD, PduP, and PduL. The bacterial microcompartment may comprise all of its endogenous Pdu proteins. For example, the bacterial microcompartment may comprise all of its endogenous Pdu proteins, with their corresponding signal sequences, and further comprise at least one of the fusion proteins described herein. Alternatively, the endogenous PduM, PduD, or any other Pdu protein of the bacterial microcompartment may be knocked out or replaced by the fusion protein described herein.

The bacterial microcompartment may comprise at least one of the signal sequences of PduD, PduP, and PduL. The bacterial microcompartment may comprise all of the signal sequences of PduD, PduP, and PduL. The bacterial microcompartment may comprise all of the signal sequences of the Pdu proteins. Each of the signal sequences may be linked to its corresponding endogenous Pdu protein, or a heterologous protein.

In a fourth aspect, provided herein is a host cell comprising at least one of the fusion proteins, compositions, constructs, or bacterial microcompartments described herein. The host cell is capable of expressing the proteins of the present disclosure. Suitable host cells include, but are not limited to, Salmonella cells. The host cell may be a Salmonella enterica cell. The host cell may be a Salmonella enterica serovar Typhimurium LT2 cell. The host cell may comprise any or all of its endogenous Pdu proteins, including PduM, PduB, and PduD. The host cell may be used to produce large quantities of the fusion protein.

In a fifth aspect, provided herein is a kit comprising at least two of the fusion proteins or polynucleotides encoding the fusion proteins described herein, wherein each fusion protein or polynucleotide is separate from the others. The fusion proteins/polynucleotides may be packaged in separate containers. The fusion proteins/polynucleotides may be lyophilized. The kit may include a buffer. The buffer may be dried with the fusion proteins/polynucleotides, and rehydrated with water. The kit may further include a dropper for dispensing controlled volumes of liquid, such as a controlled volume disposable Pasteur pipette. The kit may further include a written insert component comprising instructions for transforming a cell using the fusion proteins/polynucleotides described herein.

In a sixth aspect, provided herein is a method of targeting a microcompartment in a cell, the method comprising expressing at least one of the fusion proteins described herein in the cell. The microcompartment may be a 1,2-propanediol microcompartment. The method may comprise modifying 1,2-propanediol degradation using a fusion protein described herein wherein the heterologous cargo comprises a 1,2-propanediol pathway enzyme. The 1,2-propanediol pathway enzyme may be a heterologous or mutated enzyme having a different function or activity than the endogenous enzyme.

The microcompartment may be an ethanolamine utilization (Eut) microcompartment. The method may comprise modifying the ethanolamine utilization pathway, wherein the heterologous cargo protein comprises an enzyme of the ethanolamine utilization pathway, such as ethanolamine-ammonia lyase, aldehyde dehydrogenase (EutE), or alcohol dehydrogenase (EutG). The ethanolamine utilization pathway enzyme may be a heterologous or mutated enzyme having a different function or activity than the endogenous enzyme.

The method may comprise modifying aldehyde oxidation, wherein the heterologous cargo protein comprises an enzyme of the aldehyde oxidation pathway, such as aldehyde dehydrogenase, alcohol dehydrogenase, or phosphotransacylase. The aldehyde oxidation pathway enzyme may be a heterologous or mutated enzyme having a different function or activity than the endogenous enzyme.

The cell may be a bacterial cell. The cell may be a Salmonella enterica cell. The host cell may be a Salmonella enterica serovar Typhimurium LT2 cell. The vectors disclosed herein may be used to transform host cells. The constructs expressing the fusion proteins may be introduced to the host cells by any known method of genetic engineering, including, without limitation, recombineering and nuclease mediated genome editing (e.g., CRISPR/Cas, and A-Red-mediated recombination, among others). “Recombinecring” refers to a molecular biology technique in which PCR products and synthetic oligonucleotides are supplied as substrates, and bacteriophage-derived recombination proteins are used to recombine these oligonucleotides with homologous sites in the genome (Curr Protoc Mol Biol. (2014) 106:1.16.1-39).

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. These aspects and embodiments are illustrative and non-exhaustive in nature. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLE

Biomanufacturing has the potential to produce commodity chemicals more sustainably than traditional chemical manufacturing, but current metabolic engineering tools often cannot achieve profitable product titers. Spatial organization of enzymatic pathways is an attractive option to address this challenge, as it is used in nature by a wide range of organisms to increase the flux of metabolic pathways. For instance, over half of bacterial phyla contain bacterial microcompartments (MCPs), proteinaceous organelles that encapsulate enzymatic pathways in a semi-permeable protein shell. These pathways provide their hosts a selective advantage by enabling them to survive on niche carbon substrates that competing organisms cannot utilize. The first encapsulated step in these pathways typically produces a toxic intermediate, followed by a slow second step that detoxifies this intermediate. MCPs are hypothesized to increase pathway flux by sequestering the toxic intermediate, co-localizing enzymes to reduce diffusion limitations between steps, isolating intermediates from competing pathways, and providing a private cofactor pool.

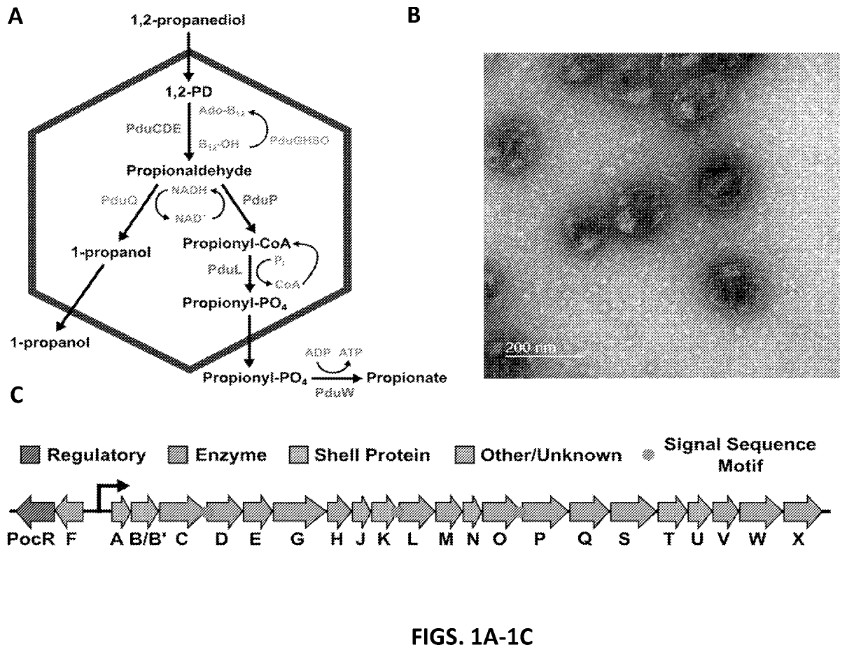

The propanediol utilization (Pdu) MCP found in Salmonella enterica serovar Typhimurium LT2 is a well-characterized MCP system that encapsulates a pathway that converts 1,2-PD to propionate and 1-propanol (FIGS. 1A and 1B). Propionate can then be utilized by the cell's central metabolism to produce energy. This pathway passes through the toxic intermediate propionaldehyde, which can cause DNA damage and slow growth if it is not sequestered in the MCP.

The proteins that form the Pdu MCP are expressed from the pdu operon in the S. enterica genome, which contains 22 genes (pduA through pduX) that are transcribed into a single mRNA strand (FIG. 1C). This operon expresses eight self-assembling proteins that form the Pdu MCP shell, pathway enzymes that convert 1,2-PD to propionate, polyhedral organelles involved in coenzyme B₁₂-dependent 1,2-propanediol degradation, cofactor recycling enzymes that regenerate Ado-B₁₂ and NADH within the MCP, and several other proteins that have not been fully characterized. Directly upstream of the pdu operon are pduF, a protein involved in transporting 1,2-PD into the cell, and pocR, a transcriptional regulator of the pdu operon. pocR is thought to interact allosterically with 1,2-PD to bind to the pdu operon promoter and activate expression.

Some MCP cargo proteins contain encapsulation peptides, or signal sequences, that target them to the enzymatic core. These signal sequences appear to target directly to the MCP core rather than to any component of the shell, as they still target to the MCP core when the core and shell are separated. Three Pdu MCP proteins contain characterized signal sequences. PduD¹⁻¹⁸ (ssPduD), Pdup¹⁻¹⁸ (ssPduP), and PduL 1-20 (ssPduL) are necessary and sufficient for encapsulation of the PduD, PduP, and PduL enzymes. These signal sequences were identified by multiple sequence alignments of PduD, PduP, and PduL with homologues not associated with compartments. The Pdu-associated enzymes had N-terminal extensions that did not occur in their homologues, suggesting that the N-termini of these proteins may have structural roles related to their compartmentalization.

In addition to mediating encapsulation of native Pdu proteins, signal sequences can also be fused to heterologous proteins to target them to the MCP core. For instance, when signal sequences are fused to fluorescent proteins, MCPs can be visualized by fluorescence microscopy as bright puncta distributed throughout the cytoplasm. This has been used to assess how the number, shape, and spatial distribution of MCPs is affected by mutations to various Pdu proteins.

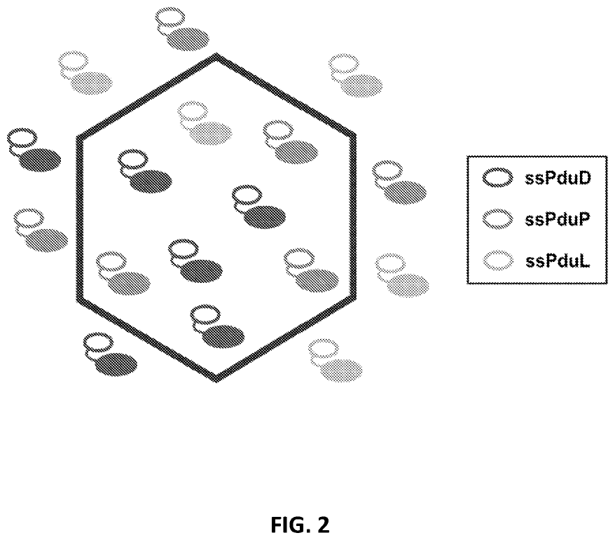

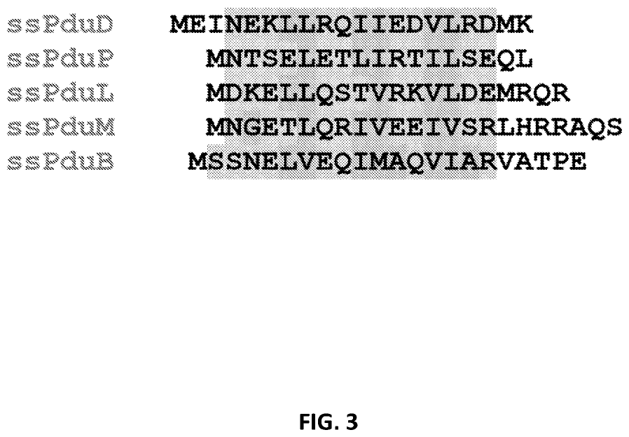

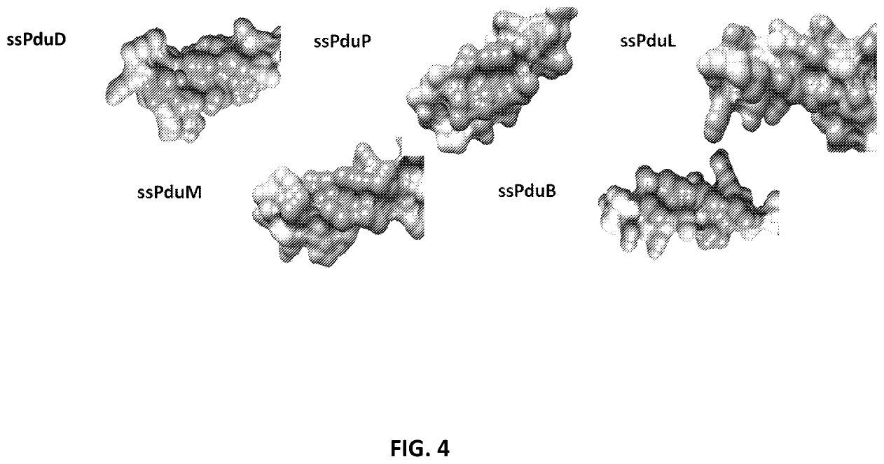

Although the amino acid sequences of these signal sequences are quite different, they share a common motif of alternating pairs of hydrophilic and hydrophobic residues (FIG. 3). This motif is widely conserved across MCP systems, and signal sequences from several non-Pdu systems have been shown to also localize to Pdu MCPs. Previous studies have suggested that signal sequences fold into α-helices with one hydrophobic side and one hydrophilic side. Signal sequence structures are illustrated in FIG. 4. While all three Pdu signal sequences share a highly conserved structure, they vary in encapsulation efficiency, which is the proportion of expressed signal sequence-tagged protein that is encapsulated in MCPs (FIG. 2). The encapsulation efficiency of ssPduD is roughly twice as high as ssPduP, which in turn is roughly three times higher than ssPduL.

MCPs are an attractive tool for metabolic engineering of enzymatic pathways that share characteristics with pathways that are natively encapsulated in MCPs, such as intermediate toxicity, kinetic bottlenecks, and high cofactor requirements. To minimize disruptions to MCP function when encapsulating heterologous pathways, it will be critical to understand how manipulating different MCP components will affect MCP assembly. Although the primary functions of most Pdu MCP components are known, how these components come together to form functional MCP shells and cores is poorly understood. Signal sequences are critical components of MCPs that are typically manipulated when encapsulating heterologous cargo, either by knocking out native signal sequences or by overexpressing signal sequences fused to heterologous proteins. Because signal sequences are responsible for targeting many cargo proteins to the MCP core, they may mediate some of the interactions involved in MCP formation and properties. We therefore investigated the structural effects of manipulating signal sequences to determine how they can be used to encapsulate heterologous cargo without disrupting MCP formation. While investigating the roles of the enzymatic Pdu signal sequences, we discovered two new signal sequences on the structural MCP proteins PduM and PduB. Because these are the first signal sequences ever discovered on structural proteins, we characterized how knockouts of these signal sequences would differ from knockouts of enzymatic signal sequences.

### Results

### Structural Effects of Knocking Out Pdu Signal Sequences

To evaluate the structural role of the enzymatic Pdu signal sequences, we combinatorially knocked out ssPduD, ssPduP, and ssPduL from the S. enterica genome. We then overexpressed a suite of encapsulation reporters in these strains. Encapsulation reporters target GFP to the MCP core by fusing it either to a signal sequence or to a cofactor recycling enzyme such as PduG or PduO, which localize to the MCP without a signal sequence. By overexpressing the ssPduD-GFP, ssPduP-GFP, and ssPduL-GFP encapsulation reporters, we could complement the signal sequence knockout strains with one of the knocked-out signal sequences.

Previous studies have used fluorescence microscopy of encapsulation reporters to show that MCP structural defects often change the number and spatial distribution of fluorescent puncta. In wild-type S. enterica, encapsulation reporters typically form three to six puncta distributed throughout the cytoplasm. When MCP assembly is disrupted such that shells either do not form or are separated from the core, encapsulation reporters typically localize to a signal fluorescent punctum located at one of the cell poles. If the encapsulation reporters no longer localize to the MCP core at all, fluorescence is evenly distributed throughout the cytoplasm of the cell.

Because signal sequences share a common structure suggested to play a role in the liquid-like behavior of the MCP core, we hypothesized that knocking out signal sequences would disrupt MCP assembly. However, if assembly defects are occurring due to lack of a signal sequence, rather than because the body of its corresponding enzyme no longer localizes to MCPs, complementing knocked-out signal sequences attached to GFP may restore proper assembly.

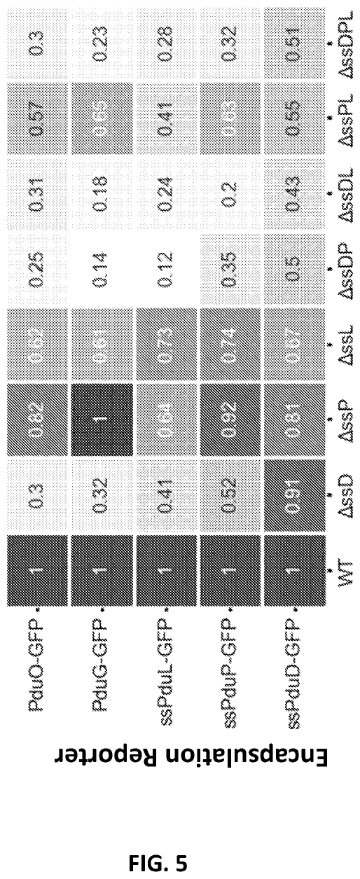

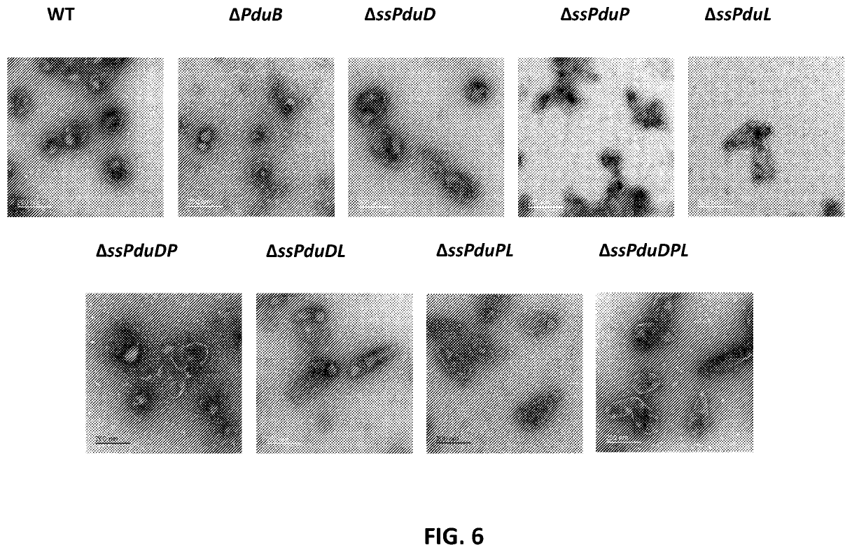

We evaluated the effects of knocking out the PduD, PduP, and PduL signal sequences (ssD, ssP, and ssL, respectively) without complementation by fluorescence microscopy of the PduG and PduO encapsulation reporters (FIGS. 5 and 6). The number of puncta per cell decreased in the knockout strains, but the spatial distribution of MCPs was not affected.

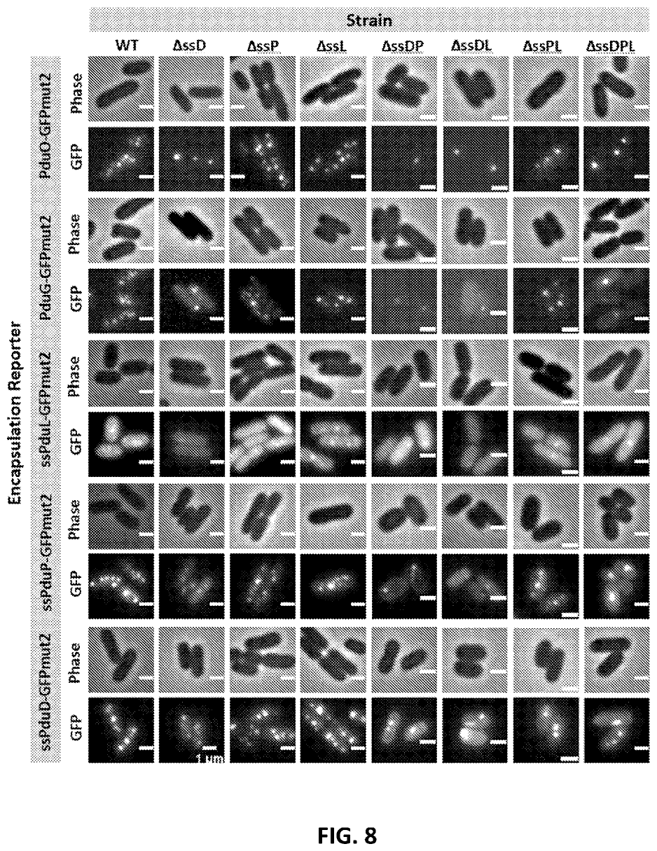

All ΔssPduD strains had an especially steep drop in puncta count (FIG. 8) and, in general, the more signal sequences that were knocked out, the larger the decrease in puncta count was. ssPduP-GFP and ssPduL-GFP showed similar patterns to PduG-GFP and PduO-GFP, but ssPduD-GFP had much lower drops in puncta count in ΔssPduD strains than all other encapsulation reporters. This suggests that overexpressing ssPduD-GFP may rescue assembly defects caused by knocking out ssPduD, but other signal sequences do not. ssPduP-GFP recovered puncta counts better than ssPduD-GFP and ssPduL-GFP in ΔssPduP and ΔssPduPΔssPduL although this pattern is less clear. ssPduL-GFP does not appear to recover puncta counts in any strains.

These patterns unexpectedly suggest that different signal sequences may support assembly by different mechanisms. Because the signal sequences share a common structure, we expected them to play the same role in MCP assembly. In addition, if decreases in MCP formation were only caused by a decrease in the amount of cargo, we would not expect that overexpressing one signal sequence would recover puncta count more than another.

We used transmission electron microscopy (TEM) of purified MCPs from each of the knockout strains to more closely evaluate changes in MCP morphology (FIG. 6). All strains formed shells and did not look like empty MCP shells, such as what occurs when PduB is knocked out (ΔPduB), indicating that they likely still encapsulated some cargo. MCPs from ΔssPduL strains were slightly elongated, which may occur due to a polar effect that decreases the expression level of the downstream pduN gene.

Overexpressing ssPduD-GFP, ssPduP-GFP, and ssPduL-GFP was done to complement the signal sequence knockout strains with one of the knocked-out signal sequences. If complementing signal sequences restored the number and spatial distribution of MCPs, this would suggest that structural defects may be occurring directly because of the lack of a signal sequence, rather than because the body of its corresponding enzyme was no longer localizing to MCPs. ssPduD-GFP only recovered puncta counts in strains without ssD. ssPduP-GFP was able to rescue puncta counts in strains without ssP. Results suggest that each signal sequence may have a different role in MCP assembly (FIG. 5). We also used transmission electron microscopy (TEM) of purified MCPs from each of the knockout strains to more closely evaluate changes in MCP morphology (FIG. 6).

### Discovery of Signal Sequences on PduM and PduB

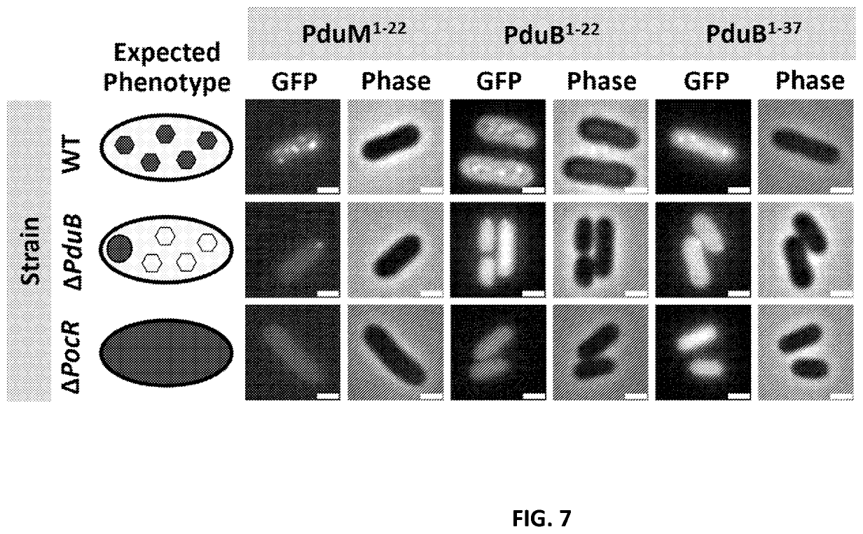

While investigating the roles of enzymatic signal sequences, we discovered two novel signal sequences on structural MCP proteins. First, we noted that the N-terminus of PduM aligns well with known signal sequences (FIG. 3). Because PduM has no known homologues outside of the Pdu MCP, this could not have been discovered from a multiple sequence alignment with non-compartment associated homologues. To test whether this motif acts as a signal sequence to target cargo to the MCP core, we fused PduM¹⁻²² to GFP and overexpressed this construct in S. enterica (FIG. 7). Like other signal sequences, PduM¹⁻²²-GFP localized to fluorescent puncta in wild-type S. enterica and to polar bodies in ApduB, indicating that it localizes to the MCP core and does not interact directly with the shell. Diffuse fluorescence was observed in ΔpocR, indicating that PduM¹⁻²²-GFP aggregation is dependent on the expression of other MCP components (FIG. 7).

Following the release of the AlphaFold Protein Structure Database, we were curious to see how the common motif shared by the Pdu signal sequences would be reflected in their predicted structures. The hydrophobicity surfaces of PduD, PduP, PduL, and PduM show that their signal sequences shared very similar structures, extending away from the body of the protein with a hydrophobic pocket on one side of the helix (FIGS. 3 and 4). Because the encapsulation mechanisms for many Pdu cargo proteins are still unknown, we searched the other proteins in the pdu operon for similar structures. Although the PduB N-terminus does not closely align with other signal sequences, AlphaFold predicts that PduB¹⁻²² folds into an amphipathic helix with a hydrophobic pocket (FIG. 4), and PduB²³⁻³⁷ is an unstructured linker between this helix and the body of PduB. Previous studies by Lehman et al. and Yang et al. have also found that PduB¹⁻³⁷ is one of the key regions of PduB required for proper connection of the MCP core and shell. This suggests that PduB¹⁻³⁷ may play a role in binding to the MCP core, similar to the function of other signal sequences. We therefore hypothesized that the N-terminus of PduB can act as a signal sequence to target heterologous cargo to MCPs.

To test this, we fused PduB¹⁻²² and PduB¹⁻³⁷ to GFP and overexpressed this construct in S. enterica. Like other signal sequences, PduB¹⁻²²-GFP and PduB¹⁻³⁷-GFP localized to fluorescent puncta in wild-type S. enterica, to polar bodies in ApduB, and were diffuse in ΔpocR (FIG. 7). However, the puncta formed by these constructs were quite dim compared to the diffuse background fluorescence, suggesting that PduB¹⁻²² may interact with MCPs more weakly than other signal sequences. This demonstrates that the N-terminus of PduB can act as a signal sequence, which raises the possibility that this region of PduB may bind the MCP shell to the enzymatic core by a similar mechanism to how other signal sequences target MCP cargo to the core.

These results suggest that encapsulation peptide activity is more a consequence of protein structure than sequence. Analysis of MCP protein structures and hydrophobicity surfaces predicted by protein folding simulations may therefore be a more accurate and comprehensive method for identifying signal sequences than searching the N- and C-termini of MCP proteins for amphipathic helices and extensions that do not occur in non-compartment associated homologues.

Characterizing the Structural Roles of ssPduM and ssPduB

Because PduM is a low-abundance structural protein, in contrast to than the high-abundance enzymatic cargo proteins encapsulated by ssPduD, ssPduP, and ssPduL, we hypothesized that assembly defects in ssPduM knockout strains would be caused by removing the body of the PduM protein from the MCP rather than the absence of the signal sequence itself. Similarly, we hypothesized that the MCP core and shell may separate in ssPduB knockout strains because PduB without the signal sequence and linker (PduB³⁷⁻²³³) can only bind to the shell and not to the core, removing the connection between the two. Therefore, we expected that knocking out ssPduM and ssPduB would yield similar assembly defects to knocking out PduM and PduB, and these assembly defects would not be rescued by overexpressing the knocked-out signal sequence.

To test these hypotheses, we overexpressed the encapsulation reporters ssPduD-GFP, PduG-GFP, ssPduM-GFP, and ssPduB-GFP in ΔssPduM, ΔssPduDΔssPduP ΔssPduLΔssPduM (ΔssPduDPLM), ΔPduM, ΔssPduB, and ΔssPduDΔssPduPΔssPduLΔssPduMΔssPduB (ΔssPduDPLMB) strains. Because PduM and PduB play roles in proper connection of the MCP shell and core, we also included a shell protein reporter, PduA-GFP, to determine how these knockouts impact shell formation.

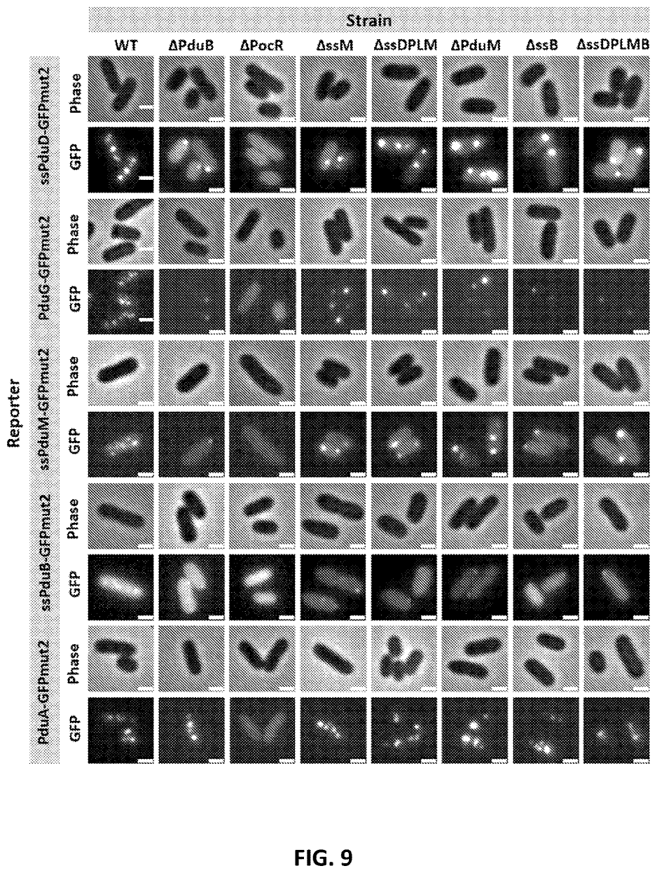

In ΔssPduM, ΔPduM, ΔssPduDPLM, all encapsulation reporters localized mostly to polar bodies, with only a few puncta distributed throughout the cytoplasm (FIG. 9). This pattern was consistent between all encapsulation reporters in these strains, indicating that overexpressing ssPduM-GFP could not recover assembly. In ΔssPduB and ΔssPduDPLMB, all encapsulation reporters localized to polar bodies, indicating that assembly was disrupted and overexpressing ssPduB-GFP could not rescue proper MCP formation. The presence of polar bodies in ΔssPduDPLMB indicates that MCP cargo still colocalize to form an MCP core without any signal sequences. This is the same phenotype observed in ΔPduB. ssPduD-GFP also could not recover assembly in ΔssPduDPLM and ΔssPduDPLMB, in contrast to ΔssPduD strains without any structural signal sequences knocked out. ssPduM-GFP and ssPduB-GFP localize in a similar pattern as other encapsulation reporters that localize to the MCP core in all strains, which indicates that PduM and PduB are likely not required for each other's encapsulation and that ssPduB may directly target the shell to the core (FIG. 9).

In strains where the core and shell are connected, the subcellular localization of PduA-GFP is similar to the encapsulation reporters. In strains where the core and shell are separated, PduA-GFP forms more puncta than the encapsulation reporters. PduA-GFP formed similar numbers of puncta in ΔssPduB, ΔPduB, and WT, showing that the MCP shell forms properly in these strains and is disconnected from the core in ΔssPduB and ΔPduB. PduA-GFP formed fewer puncta in ΔssPduDPLMB, but still more than the encapsulation reporters did. This indicates that the shell and core are still separated, but knocking out multiple signal sequences reduces the number of MCP shells that form. In ΔssPduM and ΔPduM, PduA-GFP formed fewer puncta than in WT, but still more than the encapsulation reporters did in these strains (FIG. 9). This agrees with the partial separation of the MCP core and shell observed in ΔPduM by Yang et al.

### CONCLUSIONS

We have demonstrated that the number of MCPs per cell drops as signal sequences are knocked out, particularly in ΔssPduD strains. However, overexpressing the knocked-out signal sequence attached to GFP partially recovers MCP formation in ΔssPduD) and ΔssPduP strains. These results suggest that ssPduD, and preferably all signal sequences, should be present when encapsulating heterologous pathways in MCPs, either attached to native cargo or supplemented heterologously. It remains unclear how removing signal sequences causes an assembly defect and why this defect can only be recovered by the knocked-out signal sequence, rather than any of the other signal sequences.

We have demonstrated that N-terminal extensions on two structural MCP proteins, PduM and PduB, can act as signal sequences to target heterologous cargo to MCPs. These are the first signal sequences discovered on any structural metabolosome proteins. Unlike enzymatic signal sequences, assembly defects in ΔssPduM and ΔssPduB strains could not be recovered by overexpressing the knocked-out signal sequences. This suggests that these defects are caused by removing the bodies of PduM and PduB from the MCP, rather than by removing the signal sequences themselves.

### Methods

### Recombineering

All genomic edits were generated using λ Red recombineering, a homologous recombination-based method of genetic engineering. All strains were first transformed with the pSIM6 plasmid, which contains the λ Red machinery and a carbenicillin resistance marker. Expression of the λ Red machinery is induced at 42° C. and pSIM6 is ejected from the cell at 37° C. DNA inserts were either ordered from Twist Biosciences or amplified by overhang PCR. Each insert contained about 50 bp of homology to the S. enterica genome upstream and downstream of the desired insertion site. The pSIM6 plasmid was induced at 42° C., then cells were electroporated and transformed with the desired DNA insert. Strains were recovered either at 30° C. to retain pSIM6 for future recombineering or at 37° C. to remove the plasmid.

Each genomic edit was created through two successive rounds of recombineering. In the first round, a cat/sacB cassette was inserted into the locus of interest. cat provides chloramphenicol resistance, while sacB provides sucrose sensitivity. After transformation, cells were plated on chloamphenicolto select for transformants that had successfully integrated cat sacB. In the second round of recombineering, cat sacB was replaced with the desired insert. Cells were plated on 6% sucrose to select for transformants that had successfully removed sacB. Resulting colonies were streaked onto chloramphicol plates to confirm chloramphenicol sensitivity, then sequenced at the locus of interest to confirm correct insertion of the desired mutation.

### MCP Purification

MCPs were purified from S. enterica cultures using a differential centrifugation method adapted from Sinha et al. S. enterica cultures were grown overnight in NCE media supplemented with succinate as a carbon source and 1,2-PD to induce MCP formation. After reaching an OD between 1 and 1.5, reserve samples were collected if needed, then the cultures were harvested and lysed for purification. Lysates were centrifuged at 12,000×g to remove cell debris, and the resulting supernatant was spun at 21,000×g to pellet the MCPs. The concentrations of purified MCPs were then determined by a bicinchoninic acid assay.

### Transmission Electron Microscopy

Before sample deposition, 400-mesh copper grids with a Formvar/carbon film were hydrophilized using a glow discharge system. Purified MCP samples were then deposited onto the grids and negative stained with 1% UA. Samples were imaged at 50,000× magnification using a JEOL 1400 Flash transmission electron microscope with a Gatan OneView camera.

Sequences

| ID | Description | Sequence |
|---|---|---|
| 1 | ssPduD | MEINEKLLRQIIEDVLRDMK |
| 2 | ssPduP | MNTSELETLIRTILSEQL |
| 3 | ssPduL | MDKELLQSTVRKVLDEMRQR |
| 4 | ssPduM | MNGETLQRIVEEIVSRLHRRAQS |
| 5 | ssPduB | MSSNELVEQIMAQVIARVATPE |

### REFERENCES

Bobik, T. A., Havemann, G. D., Busch, R. J., Williams, D. S., Aldrich, H. C. (1999). The propanediol utilization (pdu) operon of Salmonella enterica serovar Typhimurium LT2 includes genes necessary for formation of polyhedral organelles involved in coenzyme B12-dependent 1, 2-propanediol degradation. Journal of Bacteriology, 181(19), 5967-5975.
Chowdhury, C., Sinha, S., Chun, S., Yeates, T. O., Bobik, T. A. (2014). Diverse bacterial microcompartment organelles. Microbiology and Molecular Biology Reviews, 78(3), 438-468.
Datta, S., Costantino, N., Court, D. L. (2006). A set of recombineering plasmids for gram-negative bacteria. Gene, 379, 109-115.
Fan, C., Bobik, T. A. (2011). The N-terminal region of the medium subunit (PduD) packages adenosylcobalamin-dependent diol dehydratase (PduCDE) into the Pdu microcompartment. Journal of Bacteriology, 193(20), 5623-5628.
Fan, C., Cheng, S., Liu, Y., Escobar, C. M., Crowley, C. S., Jefferson, R. E., Yeates, T. O., Bobik, T. A. (2010). Short N-terminal sequences package proteins into bacterial microcompartments. Proceedings of the National Academy of Sciences, 107(16), 7509-7514.
Horswill, A. R., Escalante-Semerena, J. C. (1999). Salmonella typhimurium LT2 catabolizes propionate via the 2-methylcitric acid cycle. Journal of Bacteriology, 181(18), 5615-5623.
Jakobson, C. M., Tullman-Ercek, D., Mangan, N. M. (2018). Spatially Organizing Biochemistry: Choosing a Strategy to Translate Synthetic Biology to the Factory. Scientific Reports, 8(1), 1-13.
Jakobson, C. M., Kim, E. Y., Slininger, M. F., Chien, A., Tullman-Ercek, D. (2015). Localization of proteins to the 1,2-propanediol utilization microcompartment by non-native signal sequences is mediated by a common hydrophobic motif. Journal of Biological Chemistry, 290(40), 24519-24533.
Jumper, J., Evans, R., Pritzel, A., Green, T., Figurnov, M., Ronneberger, O., Tunyasuvunakool, K., . . . Hassabis, D. (2021). Highly accurate protein structure prediction with AlphaFold. Nature, 596(7873), 583-589.
Kennedy, N. W., Mills, C. E., Nichols, T. M., Abrahamson, C. H., Tullman-Ercek, D. (2021. Bacterial Microcompartments: Tiny Organelles with Big Potential. Current Opinion in Microbiology, 63, 36-42.
Kennedy, N. W., Ikonomova, S. P., Slininger Lee, M., Raeder, H. W., Tullman-Ercek, D. (2021). Self-assembling shell proteins PduA and PduJ have essential and redundant roles in bacterial microcompartment assembly. Journal of Molecular Biology, 433(2), 166721.
Kennedy, N. W., Mills, C. E., Abrahamson, C. H., Archer, A. G., Shirman, S., Jewett, M. C., Mangan, N. M., Tullman-Ercek, D. (2022). Linking the Salmonella enterica 1,2-propanediol utilization bacterial microcompartment shell to the enzymatic core via the shell protein PduB. Journal of Bacteriology, 204(9).
Kennedy, N. W., Hershewe, J. M., Nichols, T. M., Roth, E. W., Wilke, C. D., Mills, C. E., Jewett, M. C., Tullman-Ercek, D. (2020). Apparent size and morphology of bacterial microcompartments varies with technique. PLOS ONE, 15(3), e0226395.
Kim, E. Y., Jakobson, C. M., Tullman-Ercek, D. (2014). Engineering Transcriptional Regulation to Control Pdu Microcompartment Formation. PLOS ONE, 9(11), e113814.
Lee, M. S., Tullman-Ercek, D. (2017). Practical Considerations for the Encapsulation of Multi-Enzyme Cargos within the Bacterial Microcompartment for Metabolic Engineering. Current Opinion in Systems Biology, 5, 16-22.
Lehman, B. P., Chowdhury, C., Bobik, T. A. (2017). The N terminus of the PduB protein binds the protein shell of the Pdu microcompartment to its enzymatic core. Journal of Bacteriology, 199(8).
Li, Y., Kennedy, N. W., Li, S., Mills, C. E., Tullman-Ercek, D., Olvera De La Cruz, M. (2021). Computational and experimental approaches to controlling bacterial microcompartment assembly. ACS Central Science, 7(4), 658-670.
Liu, Y., Jorda, J., Yeates, T. O., Bobik, T. A. (2015). The PduL phosphotransacylase is used to recycle coenzyme A within the Pdu microcompartment. Journal of Bacteriology, 197(14), 2392-2399.
Nichols, T. M., Kennedy, N. W., Tullman-Ercek, D. (2020). A genomic integration platform for heterologous cargo encapsulation in 1,2-propanediol utilization bacterial microcompartments. Biochemical Engineering Journal, 156, 107496.
Pettersen, E. F., Goddard, T. D., Huang, C. C., Couch, G. S., Greenblatt, D. M., Meng, E. C., Ferrin, T. E. (2004). UCSF Chimera—a visualization system for exploratory research and analysis. Journal of Computational Chemistry, 25(13), 1605-1612.
Sampson, E. M., Bobik, T. A. (2008). Microcompartments for B12-dependent 1,2-propanediol degradation provide protection from DNA and cellular damage by a reactive metabolic intermediate. Journal of Bacteriology, 190(8), 2966-2971.
Sharan, S. K., Thomason, L. C., Kuznetsov, S. G. (2009). Recombineering: a homologous recombination-based method of genetic engineering. Nature Protocols, 4(2), 206.
Sinha, S., Cheng, S., Fan, C., Bobik, T. A. (2012). The PduM protein is a structural component of the microcompartments involved in coenzyme B12-dependent 1,2-propanediol degradation by Salmonella enterica. Journal of Bacteriology, 194(8), 1912-1918.
Sutter, M., Melnicki, M. R., Schulz, F., Woyke, T., Kerfeld, C. A. (2021). A catalog of the diversity and ubiquity of bacterial microcompartments. Nature Communications., 12(1), 3809.
Varadi, M., Anyango, S., Deshpande, M., Nair, S., Natassia, C., Yordanova, G., Yuan, D., . . . . Velankar, S. (2022). AlphaFold Protein Structure Database: massively expanding the structural coverage of protein-sequence space with high-accuracy models. Nucleic Acids Research, 50 (D1), D439-D444.
Yang, M., Wenner, N., Dykes, G. F., Li, Y., Zhu, X., Sun, Y., Huang, F., Hinton, J. C. D., Liu, L. N. (2022). Biogenesis of a bacterial metabolosome for propanediol utilization. Nature Communications, 13(1), 1-16.

1. A fusion protein comprising:
an N-terminal signal sequence of a Pdu protein selected from PduM and PduB; and
a heterologous cargo protein.

2. The fusion protein of claim 1, wherein the signal sequence of PduM is SEQ ID NO: 4; and the signal sequence of PduB is SEQ ID NO: 5.

3. The fusion protein of claim 1 or 2, wherein the fusion protein comprises the signal sequence of PduB.

4. The fusion protein of claim 1 or 2, wherein the fusion protein comprises the signal sequence of PduM.

5. The fusion protein of any one of claims 1-4, wherein the heterologous cargo protein comprises an enzyme.

6. The fusion protein of claim 5, wherein the enzyme is an enzyme of the 1,2-propanediol degradation pathway.

7. The fusion protein of any one of claims 1-4, wherein the heterologous cargo protein comprises a detectable protein.

8. A composition comprising two or more fusion proteins of any one of claims 1-7; wherein each fusion protein comprises a different heterologous cargo protein.

9. The composition of claim 8, wherein each fusion protein comprises the same signal sequence.

10. The composition of claim 8, wherein each fusion protein comprises a different signal sequence.

11. A construct configured to express the fusion protein of any one of claims 1-7.

12. A bacterial microcompartment comprising at least one of the fusion proteins of any one of claims 1-7.

13. The bacterial microcompartment of claim 12, wherein the microcompartment further comprises endogenous PduM and endogenous PduB.

14. The bacterial microcompartment of claim 12 or claim 13, wherein the microcompartment further comprises at least one of endogenous PduD, PduP, and PduL.

15. The bacterial microcompartment of claim 12 or claim 13, wherein the microcompartment comprises at least one of the signal sequences of PduD, PduP, and PduL.

16. The bacterial microcompartment of any one of claims 12-15, wherein the microcompartment is a 1,2-propanediol microcompartment.

17. A host cell comprising at least one of the fusion proteins of any one of claims 1-7, the composition of any one of claims 8-10, the construct of claim 11, or the bacterial microcompartment of any one of claims 12-16.

18. A kit comprising at least two of the fusion proteins of any one of claims 1-7, wherein each fusion protein comprises a different heterologous cargo protein, and wherein each fusion protein is separate from the others.

19. The kit of claim 18, wherein each fusion protein comprises the same signal sequence.

20. The kit of claim 18, wherein each fusion protein comprises a different signal sequence.

21. A method of targeting a microcompartment in a cell, the method comprising expressing at least one of the fusion proteins of any one of claims 1-7 in the cell.

22. The method of claim 21, wherein the microcompartment is a 1,2-propanediol microcompartment.

23. The method of claim 22, wherein the fusion protein modifies 1,2-propanediol degradation, and wherein the fusion protein comprises any one of the fusion proteins of claims 1-6.

24. The method of claim 21, wherein the microcompartment is an ethanolamine utilization (Eut) microcompartment.

25. The method of any one of claims 21-24, wherein the cell is a Salmonella enterica cell.
